(19) United States
(12) Patent Application Publication
(10) Pub. No.: US 2025/0098965 A1
(43) Pub. Date: Mar. 27, 2025

(54) DEVICE FOR HUMAN PERFORMANCE ASSESSMENT AND MONITORING

(71) Applicant: NOTUS LABS, INC., Avon Lake, OH (US)

(72) Inventors: Evan V. Davies, Knoxville, TN (US); Michael C. Clark, Silver Spring, MD (US); Michael B. Johnson, Sebring, OH (US)

(21) Appl. No.: 18/972,288

(22) Filed: Dec. 6, 2024

Related U.S. Application Data
(63) Continuation-in-part of application No. 17/398,111, filed on Aug. 10, 2021.
(60) Provisional application No. 63/064,153, filed on Aug. 11, 2020.

(51) Int. Cl. A61B 5/0205; A61B 5/00; A61B 5/01; A61B 5/0245

(52) U.S. Cl. CPC A61B 5/02055; A61B 5/01; A61B 5/0245; A61B 5/6802

(57) ABSTRACT

A wearable device for monitoring physiological and biomechanical parameters of a user includes a primary electronics body having a processor, a first temperature sensor, and a second temperature sensor. The first temperature sensor is in electrical communication with the processor and configured to measure a temperature of skin of the user. The second temperature sensor is in electrical communication with the processor and is configured to measure a temperature of ambient air outside the wearable device. The processor is configured to receive the measured temperatures from the first temperature sensor and the second temperature sensor, and to determine at least one metric indicative of the core temperature of the user without an invasive measurement of the body of the user.

### CROSS-REFERENCE TO RELATED APPLICATIONS

This application is a continuation-in-part of U.S. patent application Ser. No. 17/398,111, filed Aug. 10, 2021, which claims priority to and the benefit of U.S. Provisional Application No. 63/064,153, filed on Aug. 11, 2020. The entire disclosure of the above application is hereby incorporated herein by reference.

### FIELD

The present disclosure relates to monitoring devices and, more particularly, to personal health monitoring devices.

### BACKGROUND

The statements in this section merely provide background information related to the present disclosure and may not constitute prior art.

Increasing consumer interest in personal health has resulted in the development of a variety of personal health monitoring devices. Such devices have tended to be complicated to use or are typically designed for use with only one activity such as running or bicycling, for example, but not both or more than one activity. Furthermore, such devices do not provide comprehensive details about workload of a user.

Relatively recent advances in the miniaturization of sensors, power sources, and other electronics or components have enabled personal health monitoring devices to be offered in smaller sizes, form factors, or shapes than were previously feasible or industrially practical. Due to the smaller form factors, many providers are offering personal health monitoring devices that are strapped to the wrist or to the chest of the user. For example, the FITBIT VERSA LITE EDITION™ smartwatch, commercially available from Fitbit LLC in San Francisco, California, is a biometric monitoring device and smartwatch that is strapped to the wrist of the user like a traditional watch.

Undesirably, the known personal health monitoring devices that are strapped to the wrist or to the chest of the user are prone to shifting their position during strenuous athletic activities. This unintentional shifting may result in inaccurate data collection, such as inaccurate acceleration data.

There is a continuing need for a wearable monitoring device that militates against unintentional shifting during strenuous athletic activities.

Furthermore, there is a continued need for a wearable monitoring device that provides a comprehensive detail about the workload of the user, including predicting core body temperature non-invasively.

### SUMMARY

In concordance with the present disclosure, a wearable device for measuring physiological and biomechanical parameters of a user to continuously monitor a large number of metrics from a single device, which militates against unintentional shifting during strenuous athletic activities, which does not use traditional acrylate-based adhesives known to be harmful to the skin of the user, and which provides a comprehensive detail about the workload of the user including predicting core body temperature non-invasively, is surprisingly discovered.

It should be appreciated that the technology of the present disclosure is applicable to many different end users. Non-limiting examples include “weekend warriors,” patients, healthcare professionals, athletes, law enforcement officers, firefighters, armed forces service members, and industrial workers. Other suitable uses and associated end users of the technology are also contemplated and considered to be within the scope of the present disclosure.

In one embodiment, a wearable device includes a primary electronics body having a processor, a first temperature sensor, and a second temperature sensor. The first temperature sensor is attached to the primary electronics body and in electrical communication with the processor. The first temperature sensor is configured to measure a first temperature, namely, a temperature of skin of the user. The second temperature sensor is configured to measure a second temperature, namely, a temperature of ambient air outside the wearable device. The processor is configured to receive the first temperature from the first temperature sensor, and to receive the second temperature from the second temperature sensor. In operation, the processor is configured to determine at least one metric indicative of the core temperature of the user from the first temperature and the second temperature measured by the first temperature sensor and the second temperature sensor, respectively.

In one example, the wearable device further comprises an accelerometer in electrical communication with the processor. The accelerometer determines at least one of a position, orientation, and motion of the user. The processor calculates impact forces, the number of steps taken, and an external workload via the rate of change of acceleration as determined by the accelerometer, in operation.

In another example, the wearable device further includes an electrocardiogram (ECG) sensor in electrical communication with the processor. The ECG sensor may be configured to detect electrical changes of a heart of the user. In operation, the processor calculates heart rate, respiration rate, resting heart rate, and heart rate variability from the electrical changes of the heart of the user.

In yet another example, both the accelerometer and the ECG sensor are attached to the primary electronics body. In particular, the accelerometer may be attached to a center of the primary electronics body. The accelerometer is configured to determine at least one of a position, orientation, and motion of the user, and the ECG sensor is configured to detect the electrical changes of the heart of the user. The processor calculates physiological and biomechanical parameters from data collected by the accelerometer and the ECG sensor, in operation.

In various other examples, the primary electronics body is either a fully flexible circuit board, a rigid circuit board, or a combination of a flexible and rigid circuit board.

The wearable device may further have a biocompatible adhesive disposed on a bottom side of the primary electronics body. The primary electronics body or housing is thereby configured to be removably affixed to the user. In particular, the biocompatible adhesive may be a silicone-acrylate-based adhesive.

In a further example, a wearable device includes a primary electronics body having electronic components including a processor, a housing configured to enclose the electronic components, a first temperature sensor attached to the primary electronics body in electrical communication with the processor and configured to measure a temperature of skin of the user, and a second temperature sensor in electrical communication with the processor and configured to measure a temperature of ambient air outside the wearable device. The processor is configured to receive the first temperature from the first temperature sensor and the second temperature from the second temperature sensor, and to determine at least one metric indicative of the core temperature of the user from the first temperature and the second temperature.

In another embodiment, a wearable device includes a primary electronics body having electronic components including a processor, a housing configured to enclose the electronic components, a first temperature sensor, and a biocompatible adhesive. The first temperature sensor is attached to the primary electronics body and in electrical communication with the processor. The first temperature sensor is configured to measure a first temperature, namely, a temperature of skin of the user. The biocompatible adhesive is adhered on a bottom side of at least one of the primary electronics body and the housing. The housing or the primary electronics body is thereby configured to be removably affixed to the user. In particular, the biocompatible adhesive may be a silicone-acrylate-based adhesive. The processor is configured to receive the first temperature from the first temperature sensor. In operation, the processor is configured to determine at least one metric indicative of the core temperature of the user from the first temperature sensor.

In one example, the wearable device further comprises an accelerometer in electrical communication with the processor. The accelerometer determines at least one of a position, orientation, and motion of the user. The processor calculates impact forces, a number of steps taken, and an external workload via the rate of change of acceleration, in operation.

In another example, the wearable device further includes an ECG sensor in electrical communication with the process. The ECG sensor is configured to detect electrical changes of the heart of the user. In operation, the processor calculates heart rate, respiration rate, resting heart rate, and heart rate variability from the electrical changes of the heart of the user.

In another example, the wearable device further includes both the accelerometer and the ECG sensor attached to the primary electronics body. In particular, the accelerometer may be attached to a center of the primary electronics body. The accelerometer determines at least one of a position, orientation, and motion of the user. The ECG sensor is configured to detect electrical changes of the heart of the user. The processor calculates physiological and biomechanical parameters from data collected by the accelerometer and the ECG sensor, in operation.

In other various examples, the primary electronics body is either a fully flexible circuit board, a rigid circuit board, or a combination of a flexible and rigid circuit board.

In yet other examples, the wearable device further has a second temperature sensor. The second temperature sensor is configured to measure a second temperature, namely, a temperature of ambient air outside the wearable device.

In a further embodiment, a wearable device for monitoring physiological and biomechanical parameters includes a housing, a primary electronics body, and a plurality of electronic components. The electronic components are electrically connected to the primary electronics body. The plurality of electronic components includes a processor, and a memory that stores a program to be executed by the processor. The wearable device further includes a first temperature sensor in electrical communication with the processor. The first temperature sensor is configured to measure a first temperature, namely, a temperature of skin of the user. The wearable device also has a second temperature sensor in electrical communication with the processor. The second temperature sensor is configured to measure a second temperature, namely, a temperature of ambient air outside the wearable device. The wearable device also has an ECG sensor in electrical communication with the processor. The ECG sensor is configured to detect electrical changes of a heart of the user. The wearable device further has an accelerometer in electrical communication with the processor. The accelerometer is configured to measure at least one of a position, orientation, and motion of the user. The wearable device further has a biocompatible adhesive disposed on a bottom side of at least one of the primary electronics body and the housing. The primary electronics body or the housing is thereby configured to be removably affixed to the user. In particular, the biocompatible adhesive may be a silicone-acrylate-based adhesive.

In operation, the processor receives signals encoding data or information from the first temperature sensor, the second temperature sensor, the ECG sensor, and the accelerometer. The processor is configured to calculate at least one physiological and biomechanical metric from the data received, and to output at least one metric indicative of the core temperature of the user, for example to a display on the wearable device or on a graphical user interface of another device, such as a smartphone or computer, with which the wearable device is in networked communication. The location, thickness, and type of the biocompatible adhesive is configured to militate against an unintentional shifting of the wearable device on the user in operation.

Further areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way.

FIG. 1 is a bottom perspective view of a wearable device according to one embodiment of the present disclosure;

FIG. 2 is a top perspective view of the wearable device of FIG. 1;

FIG. 3 is a bottom plan view of the wearable device of FIG. 1;

FIG. 4 is an exploded cross-sectional side elevational view of the wearable device taken at section line A-A in FIG. 1;

FIG. 5 is a top plan view of a base of the wearable device with a portion of a housing removed to illustrate a printed circuit board (PCB) having a flexible portion and a rigid portion according to one embodiment of the present disclosure;

FIG. 6 is a top plan view of the base of the wearable device illustrating the flexible PCB portion and a rigid PCB portion according to the present disclosure;

FIG. 7 is a top plan view of a wearable device according to another embodiment of the present disclosure, and illustrating the use of both a rigid PCB and a flexible PCB;

FIG. 8 is a cross-sectional side elevational view of the wearable device taken at section line B-B in FIG. 7;

FIG. 9 is a side elevational view of the wearable device of FIG. 7;

FIG. 10 is a cross-sectional bottom plan view of the multifunctional device taken at section line C-C in FIG. 9, and showing an interior of the housing containing the rigid PCB;

FIG. 11 is a top plan view of the primary electronics body of the wearable device shown in FIG. 7 illustrating the flexible PCB according to another embodiment of the present disclosure;

FIG. 12 is a top view of the primary electronics body of the wearable device illustrating the flexible PCB according to yet another embodiment of the present disclosure;

FIG. 13 is a bottom plan view of a wearable device according to yet another embodiment of the disclosure, and illustrating a rigid PCB secured in the housing;

FIG. 14 is a schematic diagram illustrating various useful processes defined by executable instructions embodied in a memory of the wearable device according to certain embodiments of the disclosure, and which may be executed by the processor of the wearable device according to the present disclosure to inform the user on athletic performance and recovery;

FIG. 15 is a schematic diagram illustrating various useful processes defined by executable instructions embodied in a memory of the wearable device according to additional embodiments of the disclosure, and which may be executed by the processor of the wearable device according to the present disclosure to inform the user on detection of illness;

FIG. 16 is a front elevational view of a mobile phone with a graphical user interface (GUI) of an exemplary mobile application that can be used in combination with the wearable device according to the disclosure, the mobile phone being in networked communication with the wearable device;

FIG. 17 is a block diagram illustrating electronics components of the wearable device in electrical communication with the processor of the wearable device according to the present disclosure;

FIG. 18 is a block diagram illustrating various operating modes defined by executable instructions embodied on the memory of the wearable device, and which may be executed by the processor of the wearable device according to the present disclosure;

FIG. 19 is a schematic of a system according to one embodiment of the present disclosure; and

FIG. 20 is a schematic of a system according to another embodiment of the present disclosure.

### DETAILED DESCRIPTION

The following description of technology is merely exemplary in nature of the subject matter, manufacture, and use of one or more inventions, and is not intended to limit the scope, application, or uses of any specific invention claimed in this application or in such other applications as can be filed claiming priority to this application, or patents issuing therefrom. Regarding methods disclosed, the order of the steps presented is exemplary in nature, and thus, the order of the steps can be different in various embodiments, including where certain steps can be simultaneously performed.

The terms “a” and “an” as used herein indicate “at least one” of the item is present; a plurality of such items can be present, when possible. Except where otherwise expressly indicated, all numerical quantities in this description are to be understood as modified by the word “about” and all geometric and spatial descriptors are to be understood as modified by the word “substantially” in describing the broadest scope of the technology. The term “about” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that can arise from ordinary methods of measuring or using such parameters.

Although the open-ended term “comprising,” as a synonym of non-restrictive terms such as including, containing, or having, is used herein to describe and claim embodiments of the present technology, embodiments can alternatively be described using more limiting terms such as “consisting of” or “consisting essentially of” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application.

Disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter can define endpoints for a range of values that can be claimed for the parameter. For example, if Parameter X is exemplified herein to have value A and also exemplified to have value Z, it is envisioned that Parameter X can have a range of values from about A to about Z. Similarly, it is envisioned that disclosure of two or more ranges of values for a parameter (whether such ranges are nested, overlapping, or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X can have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it can be directly on, engaged, connected, or coupled to the other element or layer, or intervening elements or layers can be present. In contrast, when an element is referred to as being “directly on,” “directly engaged to,” “directly connected to” or “directly coupled to” another element or layer, there can be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. can be used herein to describe various elements, components, regions, layers and/or sections, these elements, components, regions, layers and/or sections should not be limited by these terms. These terms can be only used to distinguish one element, component, region, layer or section from another region, layer, or section. Terms such as “first,” “second,” and other numerical terms when used herein do not imply a sequence or order unless clearly indicated by the context. Thus, a first element, component, region, layer, or section discussed below could be termed a second element, component, region, layer, or section without departing from the teachings of the example embodiments.

Spatially relative terms, such as “inner,” “outer,” “beneath,” “below,” “lower,” “above,” “upper,” and the like, can be used herein for ease of description to describe one element or feature's relationship to another element(s) or feature(s) as illustrated in the figures. Spatially relative terms can be intended to encompass different orientations of the device in use or operation in addition to the orientation depicted in the figures. For example, if the device in the figures is turned over, elements described as “below”, or “beneath” other elements or features would then be oriented “above” the other elements or features. Thus, the example term “below” can encompass both an orientation of above and below. The device can be otherwise oriented (rotated 90 degrees or at other orientations) and the spatially relative descriptors used herein interpreted accordingly.

All documents, including patents, patent applications, and scientific literature cited in this detailed description are incorporated herein by reference, unless otherwise expressly indicated. Where any conflict or ambiguity can exist between a document incorporated by reference and this detailed description, the present detailed description controls.

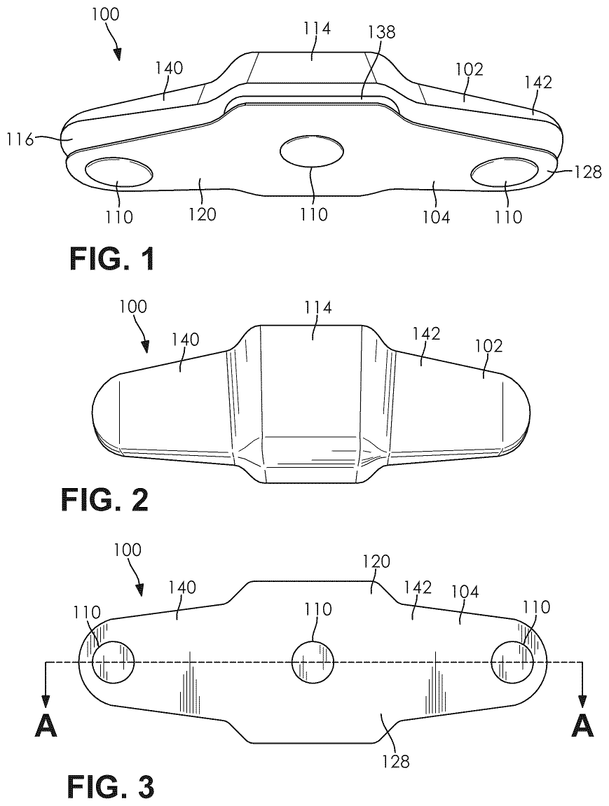

Referring to FIGS. 1-4, a wearable device 100 is shown for monitoring physiological and biomechanical parameters of a user (not shown). The wearable device 100 comprises a housing 102, a primary electronics body 104, electronic components 106, a plurality of sensors 108, at least one contact 110, and at least one layer of biocompatible adhesive 112.

The housing 102 is configured to enclose and protect the electronic components 106. The housing 102 has a top side 114 and a bottom side 116. The top side 114 forms an outer surface of the wearable device 100 when worn by the user. The bottom side 116 is disposed adjacent to the skin of the user when the wearable device 100 is being used. In certain embodiments, the housing 102 may only have the top side 114 and the primary electronics body 104 will be exposed underneath the housing 102 and configured to be disposed adjacent to the user when the wearable device 100 is worn.

The housing 102 may be formed for any of a variety of materials or combinations of materials, such as for example, a semi-flexible silicone material and/or a water-resistant material that allows the wearable device 100 to be durable and flexible enough to withstand active athletic use for a significant amount of time. It should be appreciated that other durable materials, such as other plastic or rubber materials, may also be employed while remaining within the scope of the present disclosure.

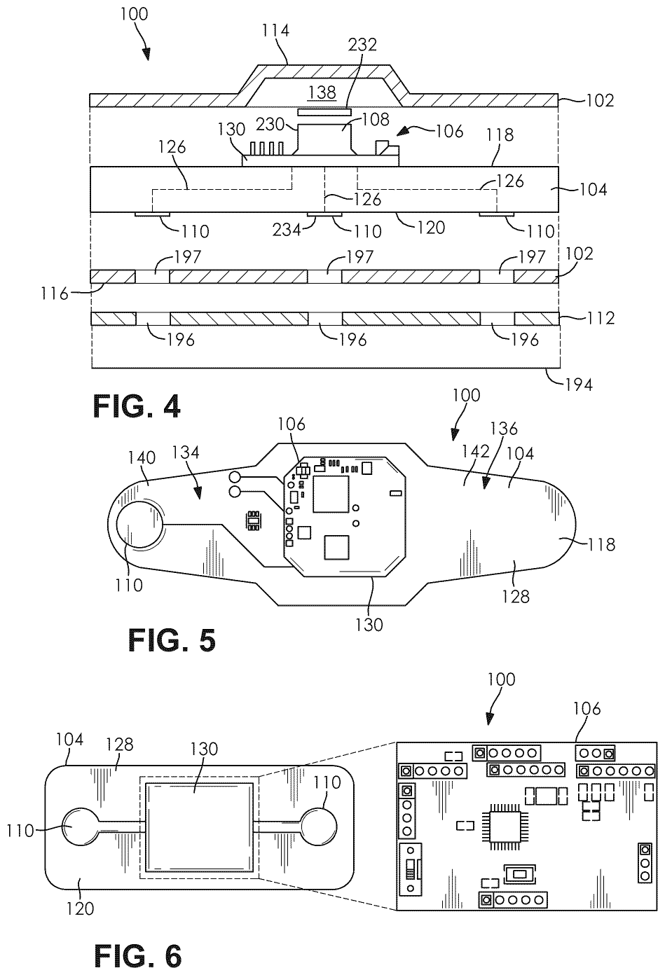

Referring to FIG. 4, the primary electronics body 104 may be defined by a printed circuit board (PCB). The primary electronics body 104 has an upper side 118, and a lower side 120. The lower side 120 that is disposed on the primary electronics body 104 opposite the upper side. The upper side 118 may be a components side on which many or all of the electronic components are attached. The upper side 118 of the primary electronics body 104 is configured to engage with an inner surface of the housing 102 to secure the primary electronics body 104 to the housing 102 such that the electronic components 106 are enclosed within the wearable device 100.

The lower side 120 may be oriented toward the skin of the user. In other words, the bottom side 116 of the housing 102 and the lower side 120 of the primary electronics body 104 may together form a side of the wearable device 100 that contacts the user in operation. In an alternative embodiment, not shown, the housing 102 may not have the bottom side 116 and the lower side 120 of the primary electronics body 104 may alone form the side of the wearable device 100 that contacts the user in operation.

The at least one contact 110 is disposed on the lower side 120 of the primary electronics body 104 and is in communication with at least one of the plurality of sensors 108. In some instances, the at least one contact 110 is in communication with at least one of the plurality of sensors 108 via a flexible trace, wire, or lead 126, for example, as shown in FIG. 4 The at least one contact 110 is configured to contact the skin of the user and serve as a measuring area for at least one of the plurality of sensors 108.

In one example, as shown in FIGS. 1-4, the at least one contact 110 is an exposed electrode pad. The exposed electrode pad, in combination with at least one of the plurality of sensors 108, may be configured to detect small electrical changes that are consequence of cardiac muscle depolarization followed by repolarization during each cardiac cycle (heartbeat). This information may be used to produce an electrocardiogram (ECG), which is described in greater detail below.

The primary electronics body 104 may also include electrical leads 126 or other layer conductors for communication between the electronic components 106 and the contact 110. As shown in FIG. 4, the leads 126 may be embedded in the primary electronics body 104, represented by dashed lines. However, it should be appreciated that one skilled in the art may position the leads 126 on upper or lower sides 118, 120 of the primary electronics body 104 while remaining within the scope of this disclosure. The leads 126 may further be covered with a protective encapsulant if positioned on the upper or lower sides 118, 120 of the primary electronics body 104.

Referring to FIGS. 5-6, in certain embodiments the primary electronics body 104 may have a flexible PCB portion 128 and a rigid PCB portion 130. The rigid PCT portion 130 may be disposed at a center 132 of the flexible PCB portion 128, for example. It should be appreciated that the flexible PCB portion 128 may permit for deformation, twisting, bending, and flexing of the primary electronics body 104, thereby allowing the wearable device 100 to conform to the curvatures of a body part of the user, such as a chest of the user, for example, while the rigid PCB portion 130 protects electronic components coupled thereto within the housing 102.

The flexible PCB portion 128 portion may include a first wing 134 disposed on one side of the rigid PCB portion 130 and a second wing 136 disposed on the other side of the rigid PCB portion 130. Without being bound to a particular theory it is believed that this configuration permits the wearable device to conform more to the curvatures of the body part of the user, such as the chest of the user, for example. It should be appreciated that although this configuration has been shown to be useful, other shapes and sizes may be employed for the flexible portion while remaining within the scope of the present disclosure.

In this embodiment, shown in FIGS. 5 and 6, the housing 102 defines a shape corresponding to the primary electronics body 104. Accordingly, the housing 102 defines a central cavity 138 forming a shell that is configured to enclose and protect the electronic components 106. The housing 102 includes a first housing wing 140 and a second housing wing 142 corresponding to the first and second wings 134, 136 of the PCB or primary electronics body 104. The first housing wing 140 is disposed on one side of the central cavity 138 and the second wing 142 is disposed on the other side of the central cavity 138. Where the housing 102 is secured to the primary electronics body 104, the first and second housing wings 140, 142 of the housing 102 align with the first and second wings 134, 136 of the primary electronics body 104, respectively, such that the electronic components 106 utilizing the rigid PCB portion 130 are enclosed within the central cavity 138 of the housing 102.

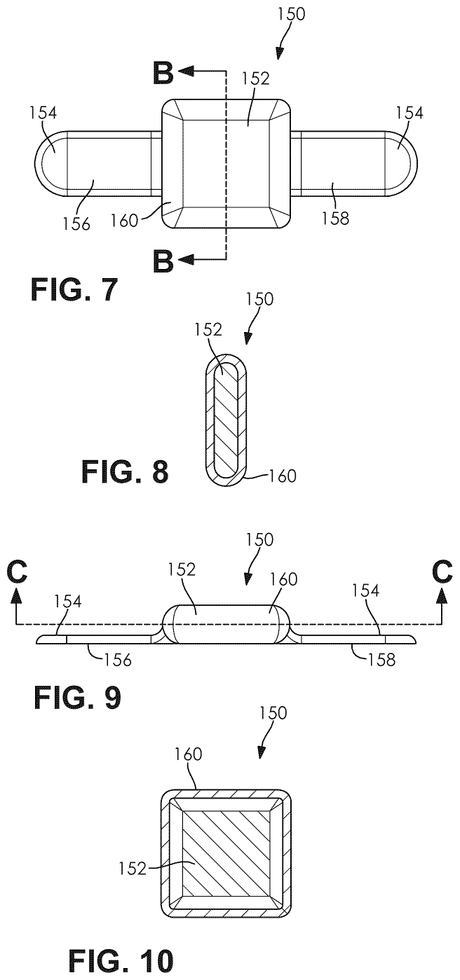

Referring to FIGS. 7-10, a wearable device 150 according to another embodiment of the present disclosure is illustrated. In this example, the wearable device 150 includes a rigid PCB portion 152 and a flexible PCB portion 154. The flexible PCB portion 154 is defined by a first flexible wing 156 disposed on one side of the rigid PCB portion 152 and a second flexible wing 158 disposed on the other side of the rigid PCB portion 152. The rigid PCB portion 152 is disposed within an electronics housing 160, which is configured to enclose and protect electronic components 162. The electronics housing 160 encapsulates the rigid PCB portion 152; however, the electronics housing 160 does not extend beyond the rigid PCB 152. Accordingly, and unlike the embodiments shown in FIGS. 1-6, much of the flexible PCB portion 154 is not in contact with the housing 160 in the embodiment shown in FIGS. 7-10.

With reference to FIG. 7, it should be appreciated that the dimensions of the wearable device 150 may be selected depending on the desired body part and location of measurement for the end user. For example, the wearable device 150 may have a maximum length and a maximum width, with the maximum length being at least three times the maximum width of the wearable device 150. In specific examples, the maximum length may be around three inches (3″) and the maximum width may be around one inch (1″). While these dimensions have shown to be useful, one skilled in the art may select other dimensions within the scope of this disclosure.

With reference to FIGS. 7 and 9, the electronics housing 160 may have a housing length, a housing width, and a housing height. The housing length and the housing width may be selected to be about the same, in certain cases, and may about at least three times the housing height. In specific examples, the housing length may be around one inch (1″), the housing width may be around one inch (1″), and the housing height may be about one-third of an inch (0.31″). However, it should be appreciated that a skilled artisan may employ different but suitable dimensions for the electronic housing, as desired.

The flexible PCB portion 154 may further be flexible in more than one dimension. For example, the flexible PCB portion 154 may have at least one of a flexible length, flexible width, and a flexible height. In specific examples, the flexible length is about two inches (2″). Also, the flexible width is about one-half of an inch (0.50″). In addition, the flexible height is less than about one-tenth of an inch (0.06″). Although these dimensions have shown to be useful, other dimensions may be chosen for the flexible PCB portion 154, as desired.

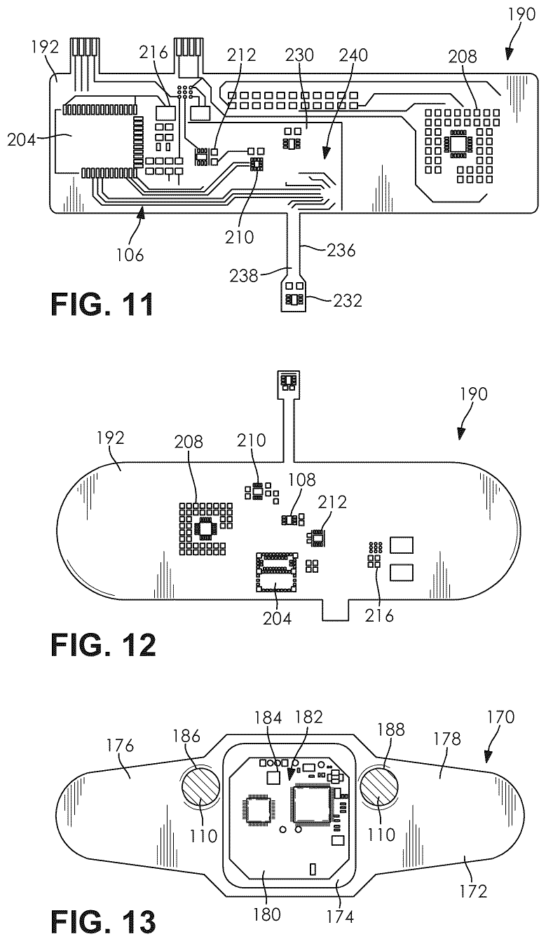

Referring to FIGS. 11-12, a wearable device 190 according to another embodiment of the present disclosure is illustrated. In this form, the wearable device 190 includes a primary electronics body 192 that may be defined by a fully flexible PCB. A geometry of the flexible PCB or primary electronics body 192 may be in the shape of a rectangle (FIG. 11) or a stadium (FIG. 12); however, other shapes may be employed while remaining within the scope of the present disclosure.

Referring to FIG. 13, a wearable device 170 according to yet another embodiment of the present disclosure is illustrated. The wearable device 170 includes a housing 172 defining a central aperture 174, a first wing 176 disposed on one side of the central aperture 174 and a second wing 178 disposed on the other side of the central aperture 174. The wearable device 170 further includes a rigid PCB 180, electronic components 182 attached to the rigid PCB, and a battery 184 disposed within the central aperture 174 of the housing 172 to protect the battery 184 and rigid PCB 180. Each one of the first and second wings 176, 178 defines an opening 186, 188 having the at least one contact 110 placed adjacent thereto.

With renewed reference to FIG. 4, the at least one layer of biocompatible adhesive 112 is configured to be removably affixed to the body part of the user, such as the chest of the user, in operation. Advantageously, removably affixing the at least one layer of biocompatible adhesive 112 to the chest of the user facilitates the wearable device 100 to remain in substantially the same position on the chest of the user. This may result in more accurate readings from the plurality of sensors 108 since the employment of the biocompatible adhesive 112 militates against the at least one contact 110 losing connection to the skin of the user. In addition, the at least one layer of the biocompatible adhesive 112 militates against an unintentional shifting during strenuous athletic activities, unlike traditional wearables that are strapped to the wrist or the chest of the user with belts or bands. It should be appreciated that one skilled in the art may scale the location, type, and dimensions of the layer of biocompatible adhesive 112, as desired.

In specific examples, the at least one layer of the biocompatible adhesive 112 is a silicone-acrylate-based adhesive. Without being bound to a particular theory, it is believed that that the silicone-acrylate-based adhesive alleviates some of the known irritations brought about by some commercial acrylate-based adhesives. Desirably, the silicone-acrylate-based adhesive also facilitates a long-term duration of adhesion to the body part, such as the chest, of the user. It should be appreciated that a skilled artisan may select other biocompatible adhesives while remaining within the scope of the present disclosure.

In another example, the at least one layer of the biocompatible adhesive 112 is a double-sided adhesive having a first side removably attached to the wearable device 100 and a second user side configured to be removably affixed to the chest of the user. Suitable silicone-acrylate-based double-sided adhesive may include 2477P™ Double Sided Silicone/Acrylate Thermoplastic Elastomer, commercially available from 3M; however, other suitable types of silicone-acrylate-based adhesives are contemplated and may be selected by one of ordinary skill in the art within the scope of the present disclosure.

It should be appreciated that the biocompatible adhesive 112 may be either permanently affixed or removably affixed to at least one of the housing 102 and the primary electronics body 104, as desired. Where the biocompatible adhesive 112 is removably affixed, the primary electronics body 104 may include fasteners (not shown) for removably holding the biocompatible adhesive 112. Additionally, where removably affixed, it should be appreciated that the biocompatible adhesive 112 may be replaced for repeated use of the wearable device 100 long-term. For example, where removably affixed, the biocompatible adhesive 112 may be removed and replaced before each subsequent use.

Regardless of whether the biocompatible adhesive 112 is permanently or removably affixed, it should be understood that a protective backing 194 (shown in FIG. 4), such as a peel away plastic film may also be employed on the user side until adherence of the wearable device 100 to the skin is desired.

The at least one biocompatible adhesive 112 may also define at least one opening 196 having a geometric shape corresponding to that of the at least one electrode or contact 110. The at least one opening 196 allows the electrode or contact 110 to directly contact the skin, in operation. It should be appreciated that there may at least one corresponding hole 197 also formed in the housing 102, for example, as shown in FIG. 4. The corresponding hole 197 alights with the at least one opening 196 of the adhesive 112 to allow the electrode or contact 110 to be disposed therethrough. The biocompatible adhesive may further incorporate a gel electrode material for use in monitoring vitals in electrocardiogram (ECG) applications, which is described in greater detail below.

As shown in FIGS. 4 and 11, the electronic components 106 are electrically connected to the primary electronics body 104 and coupled to the at least one contact 110 and/or one of the plurality of sensors 108 to measure physiological and biomechanical information for continuous monitoring of a large number of metrics from a single device.

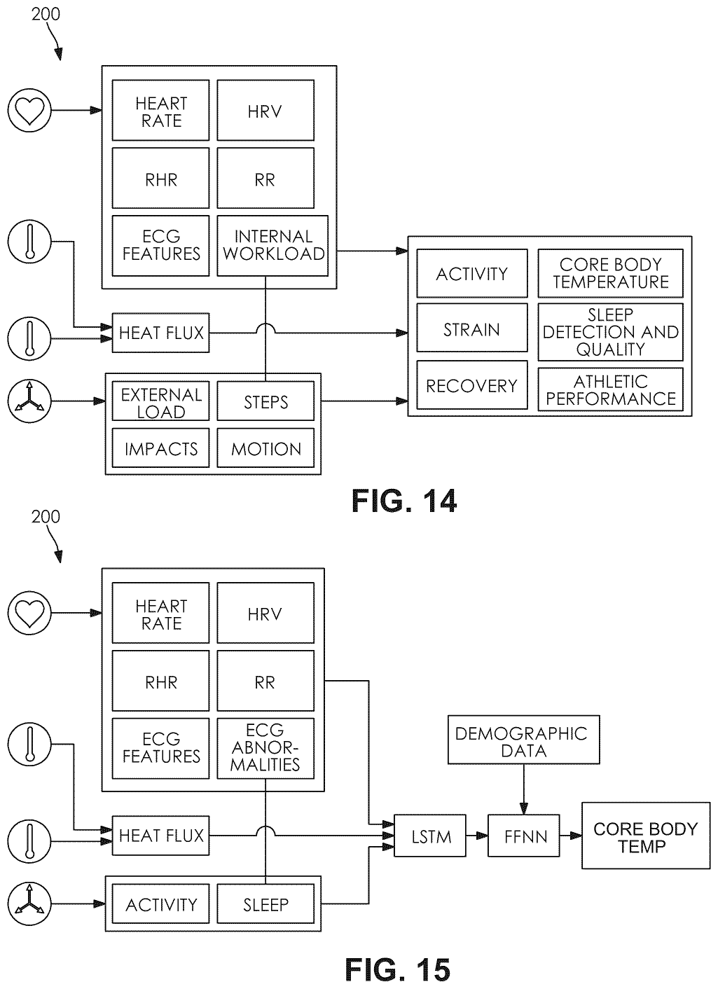

Referring to FIGS. 14-15, the wearable device 100, 150, 170, 190 may utilize an analytical software platform 200. The analytical software platform 200 is configured to convert the raw physiological and biomechanical measurements into relevant metrics for the user. Non-limiting examples of the relevant metrics includes at least one of heart rate (HR), respiration rate (RR), heart rate variability (HRV), resting heart rate (RHR), ECG features, skin temperature, ambient temperature, heat flux, acceleration in X, Y, Z axes, impacts, steps, external workload, sleep, activity, recovery, motion profiling, cardiovascular strain, internal workload, athletic performance, core body temperature, and the probability of infection or overall wellness. Examples of the ECG features may include, but are not limited to, PQRST and U wave location, amplitude, phase and area, and time difference and length of all waves and wave complexes, for example, QRS, QT interval, and PQ interval, among others.

It should be appreciated that the relevant metrics may include also include other information relevant to the health of the user, as desired.

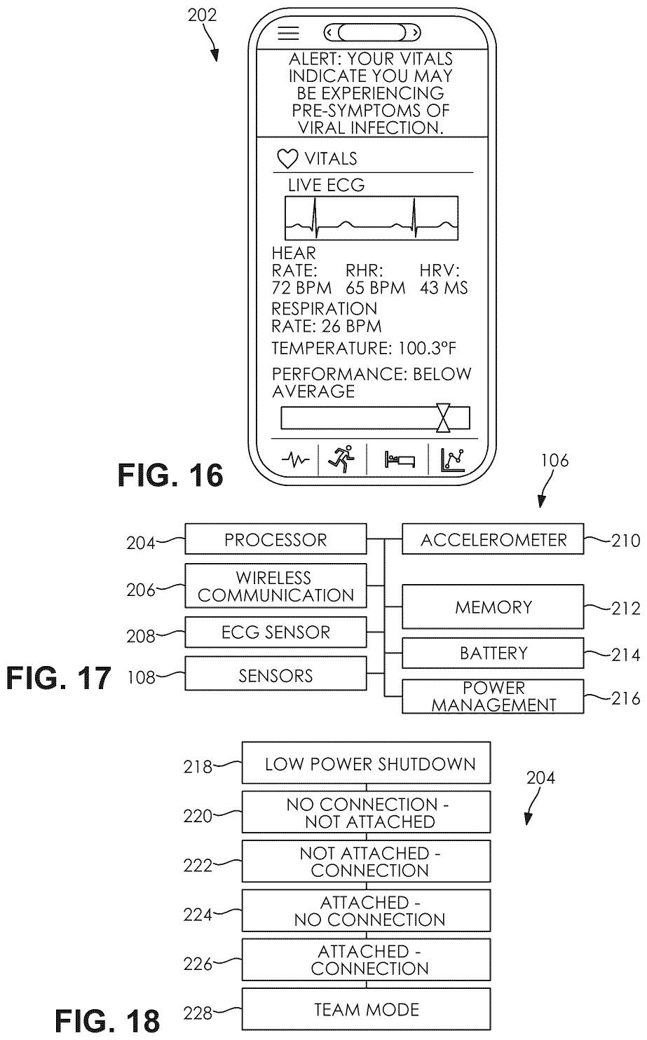

The analytical software platform 200 may be defined by processor-executable instructions stored on a memory of the wearable device 100, 150, 170, 190. In other instances, the analytical software platform 200 is a part of a separate application 202 on a mobile device (shown in FIG. 16) or networked computer that is used in combination with the wearable device 100, 150, 170, 190. Non-limiting examples of the separate application may include a web app, a dedicated application, or a mobile application. It should be appreciated that the software may take the form of other types of applications, within the scope of this disclosure.

The analytical software platform 200 is also configured to perform several different processes with data collected by the wearable device 100, 150, 170, 190 such as, but not limited to, data collection, signal processing, and use of early detection algorithms. The early detection algorithms may be utilized to calculate the probability of infections. Desirably, this may allow users to identify illnesses and other infections within their bodies before symptoms occur, and receive warnings about heat illness, heart conditions, and soft tissue injuries.

In specific examples, as shown in FIG. 15, the analytical software platform 200 may further include neutral networks such as Long Short-Term Memory (LSTM) networks. LTSM networks are configured to facilitate recognizes long term dependences. Advantageously, this allows LSTM networks to assist in predicting the outcome of a sequence of time dependent data. It should be appreciated that a skilled artisan may employ other structure and features within the analytical software platform 200, such a feedforward neural network (FFNN), machine learning, and artificial intelligence (AI), as desired.

Referring to FIGS. 11 and 17, the electronic components 106 include a processor 204, at least one wireless communication protocol 206, an ECG sensor 208, the plurality of sensors 108, an accelerometer 210, a memory 212, a battery 214 and a power management system 216.

Other electronic components such as transmitters, receivers, or transceivers (not shown) for transmission and receipt of wireless radio signals may also be provided in communication with the processor 204. For example, the processor 204 in communication with the transceiver is capable of processing, receiving, and transmitting data or instructions. The processor 204 is configured to access a memory 212 having a tangible, non-transitory storage medium on which processor-executable instructions are embodied. The processor-executable instructions may define one or more programs configured to be executed by the processor 204. The one or more programs may include instructions configured to perform one or more of the operations or functions described herein with respect to the wearable device 100, 150, 170, 190. For example, the instructions may be configured to control or coordinate one or more communication channels and collect data from the plurality of sensors, perform signal processing, communicate with external devices, and control the overall operations of the wearable device, within the scope of the present disclosure.

The memory 212 can further store electronic data that can be used by the wearable device 100, 150, 170, 190. For example, the memory 212 can store electrical data such as timing and control signals or data for various modules, data structures or databases. The memory 212 can be any type of memory, for example, the memory 212 can be implemented as a read only memory (ROM), electrically erasable programmable read only memory (EEPROM) and/or random-access memory (RAM). One skilled in the art may also select other suitable types of technology for the memory 212, as desired.

In one non-limiting example, the processor 204 comprises a memory 212 that includes a non-transitory computer readable storage medium that stores a program configured to be executed by the processor 204. The program comprises instructions, which when executed by the processor 204 causes the wearable device 100, 150, 170, 190 to: i) receive physiological measurements representative of an internal load of the user detected by skin temperature sensor and/or a ECG sensor; ii) receive biomechanical measurements representative of an external load of the user detected by the ambient temperature sensor, magnetometer, and/or the accelerometer; and iii) determine at least one metric indicative of the users health and/or performance.

The at least one wireless communication protocol 206 is adapted to provide communication between the processor 204, by way of the transmitter, receiver, or transceiver, and an external device, such as a mobile phone, computer, tablet, or the like. The at least one wireless communication protocol 206 may be configured to transmit and receive data or signals that may be interpreted by the instructions on the processor 204. In a specific example, the wearable device 100, 150, 170, 190 communicates with an external device using Bluetooth Low Energy Protocol (BLE). However, it should be appreciated that one skilled in the art may use other wireless communication protocols, such as for example, ANT, Zigbee, LoRa and/or LoRaWAN, while remaining within the scope of the present disclosure.

The battery 214 is adapted to store and provide power to the components of the wearable device 100, 150, 170, 190. The battery 214 may be a rechargeable power supply configured to provide power to the wearable device while it is being worn by the user. The wearable device 100, 150, 170, 190 may be configured to recharge the battery 214 using wireless charging via an external charging pack to let users recharge the wearable device without taking it off. Alternatively, the device may be configured for wired charging. As such, the wearable device 100, 150, 170, 190 includes a power management system 216 that receives power from an external device, such as for example an external charging pack, and is configured to deliver power to the electronic components 106, including the battery 214.

As discussed above, the wearable device 100, 150, 170, 190 may be made of a water-resistant material that allows the wearable device 100 to be durable and flexible enough to withstand active athletic use for a significant amount of time. To maintain a fully waterproof wearable device, the charging pack and wearable device 100, 150, 170, 190 are equipped with wireless charging capabilities.

Referring to FIG. 18, the processor 204 may operate in various modes, as defined by the processor-executable instructions on the memory 212, and based on a status of the wearable device 100, 150, 170, 190, such as, for example, power level status of the battery 214 and/or a status of a connection with an external device. Non-limiting examples of different operating modes may include:

### A. Low Power “Shutdown” Mode 218.

When the processor 204 detects the battery 214 is running out of power, the processor 204 will initiate Low Power “Shutdown” Mode to preserve as much power as possible until the battery 214 is recharged. This mode protects the battery 214 from degradation. In this mode, the plurality of sensors 108 are disconnected from the power supply, no wireless communication is available, and the processor 204 will enter a “sleep” mode where the processor operates simply to occasionally check if the battery has been recharged.

### B. No Wireless Connection, not Attached to User 220.

When the wearable device 100, 150, 170, 190 detects it is not attached to the user and there is no wireless connection to an external device, the wearable device will enter this mode. In this mode, the processor 204 disconnects a majority of the plurality of sensors 108 from the power supply, and occasionally checks if the wearable device has been attached to the user or if a wireless connection has been requested.

### C. Not Attached to User, Wireless Connection 222.

When the wearable device 100, 150, 170, 190 establishes a wireless connection has been made while the wearable device is not attached to the user, the wearable device will communicate with the external device but readings from the plurality of sensors 108 must be manually requested by the external device. This mode is primarily used for testing, but it can also be used for device firmware update (DFU) and collecting data saved from the memory 212.

### D. Attached to User, No Wireless Connection 224.

When the wearable device 100, 150, 170, 190 detects it is attached to the user but there is no wireless connection to an external device, it enters this mode. In this mode, the processor 204 continuously reads data collected from the plurality of sensors, performs signal processing, and stores the processed signals in the memory 212 until a wireless connection is established.

### E. Attached to User, Wireless Connection 226.

This is the normal operating mode for the wearable device. In this mode, the processor continuously reads data collected from the plurality of sensors 108, may perform signal processing, and streams the raw or processed data to the external device. The wearable device 100, 150, 170, 190 can also be updated through DFU in this state. In this state, any data stored in the memory 212 from a period without a wireless connection is simultaneously streamed to the connected external device.

### F. Team Mode 228.

When the wearable device 100, 150, 170, 190 is worn by a group of users, the users have an option to form a team. When operating in a team mode, the wearable devices will form a mesh network. The mesh network transmits data from one device to another across the mesh to either a central collection and analysis location, such as an athletic trainer, coach, and/or team physician, or to one or more team members' individual mobile phone. This allows teams to compete or train together while still receiving continuous monitoring from the sidelines of a field without requiring members of the team to have their mobile devices with them.

The ECG sensor 208 includes at least one electrode or contact 110 configured to detect electrical changes that are consequence of cardiac muscle depolarization followed by repolarization during each cardiac cycle. As shown in FIGS. 3-4, three electrodes or contacts 110 may be affixed to the lower side 120 of the primary electronics body 104 and placed in direct contact with the chest of the user when the wearable device 100 is attached. Cardiac related metrics such as heart rate (HR), resting heart rate (RHR), heart rate variability (HRV), and respiration rate (RR) are calculated through algorithms that first identify each ECG feature associated with ECG signal. The ECG features may include, but are not limited to, PQRST and U wave location, amplitude, phase and area, and time difference and length of all waves and wave complexes, for example, QRS, QT interval, and PQ interval, among others.

The HR, RHR, HRV, and RR may be calculated using just the R wave. For example, the HR is calculated in beats per minute (BPM) from the number of R waves recorded over time. RHR is calculated in BPM measured during certain stages of sleep when the user is inactive. HRV is calculated by recording the time in milliseconds between R waves and the average time variation between beats. RR is measured from the ECG signal due to Respiratory Sinus Arrhythmia.

The plurality of sensors 108 comprises a first temperature sensor 230 configured to measure a temperature of the skin of the user and a second temperature sensor 232 configured to measure a temperature of ambient air outside the wearable device 100, 150, 170, 190. As shown in FIGS. 4 and 11, the first temperature sensor 230 may be positioned directly above the skin of the user on the upper side 118 of the flexible primary electronics body 104 that forms the bottom of the wearable device 100, 150, 170, 190. In this example, a skin connection 234 may be made when the least one contact 110 touches the skin of the user. As a nonlimiting example, the at least one contact 110 may be a pad of copper printed on a bottom side 120 of the flexible PCB directly below the first temperature sensor 230 to facilitate heat transfer into the first temperature sensor 230.

As also shown in FIGS. 4 and 11, the second temperature sensor 232 may be connected to the wearable device 100, 150, 170, 190 by a long section of the flexible PCB 236 (FIG. 4). The second temperature sensor 232 may be positioned on a top side 238 of the long section of the flexible PCB 236, and a copper layer may be printed on a bottom layer of the long section of the flexible PCB 236 directly below the second temperature sensor 232 for increased heat transfer into the second temperature sensor 232. The long section 236 of the flexible PCB may be folded above the electronic components 106 on the upper side 118 of the flexible primary electronics body 104 and incorporated into the housing 102, for example, as shown in FIG. 4.

The first and second temperature sensors 230, 232 are configured to record and average values continuously and return a digital temperature measurement with accuracy. In a specific example, the first and second temperature sensors 230, 232 record and average values continuously over a 1 second sampling period and return a 16-bit resolution digital temperature measurement with better than ±0.1° C. accuracy.

The skin temperature measurements may be read directly from the wearable device 100, 150, 170, 190 and heat flux is measured through a comparison of skin and ambient temperature measurements, which has been surprisingly found to beneficially contribute to a more reliable and non-invasive prediction of core body temperature.

Referring to FIG. 11, the accelerometer 210 may be disposed near a center 240 of the flexible PCB or primary electronics body 192. The accelerometer 210 may be configured to measure acceleration forces acting on the user to determine the position, orientation and/or motion of the user. The accelerometer 210 can measure static acceleration forces, for example, gravitational force, and/or dynamic acceleration forces, for example, forces caused by movement or vibration of the accelerometer.

Although the wearable device 100, 150, 170, 190 is shown in FIG. 11 as having one accelerometer 210, it should be appreciated that more than one accelerometer or an array of multiple accelerometers may be employed to monitor acceleration at several locations on the wearable device while remaining within the scope of the present disclosure.

The accelerometer 210 can be an AC-response accelerometer (for example, charge mode piezoelectric accelerometer, voltage mode piezoelectric accelerometer), a DC-response accelerometer (for example, capacitive accelerometer, piezoresistive accelerometer), a microelectromechanical system (MEMS) accelerometer, or the like. The accelerometer can measure acceleration forces in one-dimension, two-dimensions, or three-dimensions. Any number of accelerometers can be used collect sufficient data to determine position and/or movement of the user while remaining within the scope of the present disclosure.

The accelerometer 210 can measure and output signals related to a linear acceleration of the user with respect to gravity along three axes. The first axis, or “roll,” corresponds to a longitudinal axis of and/or extending through the body of the user, such as along a length and/or height of the user. Accordingly, the roll measurement can be used to determine whether the user is in a prone position, a supine position, or on a side. The second axis, or “pitch,” of the accelerometer corresponds to the locations about the hip of the user, such as an axis extending between and/or through the hips of the user. The pitch measurement can be used to determine whether the user is sitting up or lying down. A third axis, or “yaw,” of the accelerometer corresponds to a horizontal plane in which the user is located. The three axes that the accelerometer can measure linear acceleration are referred to as the X, Y, and Z axes.

In one specific example, the accelerometer 210 is a tri-axial accelerometer having a digital output that includes three signals representing measured acceleration along a particular axis. In this example, the output of the accelerometer is 16-bit, however, any sized output signal, such as for example 8-bit or 12-bit may be incorporated while remaining within the scope of the present disclosure. The range of acceleration measured is set from ±2 g to ±16 g.

The accelerometer 210 can be used to determine the position, orientation, and/or motion of the user, which allows for recording or calculating parameters such as impact forces, the number of steps the user takes, and an external workload via the rate of change of acceleration. The users step count can be used to measure other physical activities such as total calories burned, or distance covered. Furthermore, a motion profile of the user is important to the development of enhanced training programs that are tailored to specific goals of the user, such as an athlete, to maximize performance and reduce injury.

With renewed reference to FIGS. 14-15, a large number of physiological and biomechanical metrics are recorded or calculated from the data collected by the plurality of sensors, directly or indirectly through computations. The use of multiple sensors allows the wearable device to give a comprehensive detail about the health and fitness of the user.

Metrics directly recorded or calculated from data received by the plurality of sensors include direct ECG metrics, direct temperature metrics, and direct accelerometer metrics. Direct ECG metrics include HR, RR, HRV, RHR, ECG features, for example PQRST and U waves, and time difference and length of all waves and wave complexes. Direct temperature metrics includes skin temperature, ambient temperature, and heat flux. Direct accelerometer metrics include acceleration in X, Y, Z axes, impacts, steps, and external workload.

The above stated metrics can be combined to provide further physiological and biomechanical metrics to be recorded or calculated from the data collected by the plurality of sensors indirectly through computations. Non-limiting examples of indirect metrics include sleep detection and quality, activity, recovery, motion profiling, cardiovascular strain, internal workload, athletic performance, and core body temperature.

The sleep metrics are derived from HR, RR, acceleration, and temperature metrics. Activity metrics are derived from HR, acceleration, and temperature metrics. Recovery metrics are derived from HR and activity metrics. Motion profiling is derived from acceleration, cardiovascular strain and internal workload are derived from ECG metrics. Athletic performance is derived from internal workload and external workload.

Core body temperature is derived from HR, temperature, and heat flux. When core body temperature is paired with HR and ECG information, internal exertion can be calculated, which then allows user to measure how heat is getting dissipated. Predicting core body temperature lets a user know when their body temperature is approaching overheating.

In a further example, cardiovascular information, temperature information, and accelerometry can calculate how hard the internal body is working compared to how much work is being produced externally. When the two are compared, you can determine how much you respond to a certain level of external activity and quantify fitness objectively.

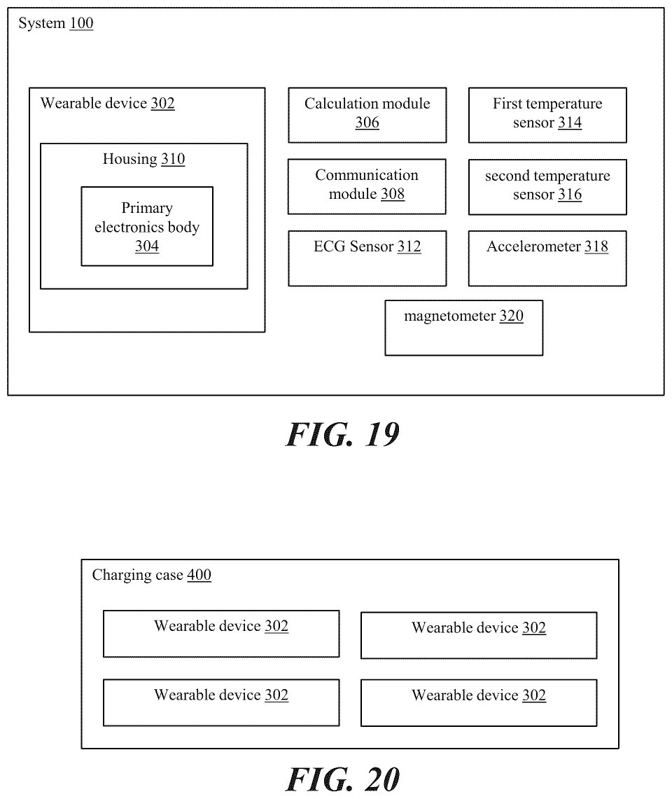

With reference to FIG. 19, certain embodiments of the present disclosure relate to a system 300 configured for monitoring physiological and biomechanical parameters of a user, incorporating a wearable device 302 equipped with various sensors and electronics, integrated into a monitoring framework that facilitates real-time data processing, transmission, and analysis. The system 300 can be particularly advantageous for athletes and individuals engaged in physical activities, aiming to enhance performance and safety through continuous monitoring.

Embedded within the wearable device 302 is a primary electronics body 304. A calculation module 306 can be operably connected to the primary electronics body 304. The calculation module 306 can utilize algorithms for calculating core body temperature, as well as determining external workload and energy expenditure based on data from sensors as described in greater detail herein.

A communication module 308 of the system 100 can be operably connected to the primary electronics body 304 and the calculation module 306. The communication module 308 can support robust data transmission capabilities, enabling continuous communication between the sensors of the wearable device 302, the calculation module 306, and external systems such as mobile devices, computers, or cloud-based platforms. The communication module 308 can be configured for the real-time transmission of physiological and biomechanical data to external devices for immediate monitoring, allowing coaches, trainers, and medical staff to make informed decisions quickly.

In the disclosed system 300, the calculation module 306 can be integrated directly within the wearable device 302 or configured remotely, depending on specific operational requirements or desired functionalities. When located on the device 302, the modules facilitate immediate data processing and real-time feedback. Alternatively, positioning the calculation module 306 remotely, such as in a cloud-based server or a connected mobile device, allows for leveraging greater computational power and expansive storage capabilities.

The system 300 includes the wearable device 302 that can have a housing 310. The housing 310 can be configured to be compact and unobtrusive, enhancing the comfort and usability for users during performance. The dimensions of the housing 310 can be tailored to balance the need for containing all necessary electronics while maintaining a low profile on the user. In certain non-limiting embodiments, the housing 310 can have a length between approximately 25 mm and 35 mm, a width between approximately 20 mm and 35 mm at the shortest, and a height between approximately 5 mm to 15 mm. As a specific non-limiting example, the housing 310 can have a length of approximately 31 mm, a width of approximately 27 mm, and a height of approximately 9.5 mm in height. These dimensions are provided as non-limiting examples, and a skilled artisan can select other suitable dimensions within the scope of the present disclosure.

The housing 310 can be affixed to a chest of the user with an adhesive component, in particular, a left side of the chest of the user. The adhesive component can be configured to provide both a secure attachment and functionality. The adhesive component can include two different adhesive materials. The first adhesive material, which contacts skin of the user, can be a medical-grade adhesive. One non-limiting example can be 4076 SC Spunlace Extended Wear Nonwoven Tape, which is available commercially from 3M Medical Materials & Technologies, having offices in St. Paul, Minnesota. The second adhesive material, used on the housing side, can be Medical Tape 1509, Double Sided Transparent Polyethylene, which is available commercially from 3M Medical Materials & Technologies, having offices in St. Paul, Minnesota.

The wearable device 302, in particular the electronics body 304, can be in communication with a plurality of sensors configured to monitor various physiological and biomechanical parameters of the user, enhancing both athletic performance and safety. The sensors can include an electrocardiogram sensor 312, a first temperature sensor 314, a second temperature sensor 316, a three-axis accelerometer 318, and a three-axis magnetometer 320.

The electrocardiogram sensor 312 is configured for monitoring the electrical activity of the heart. A dry electrode can be embedded within the adhesive component of the housing 310 and allows for the effective transmission of the electrocardiogram (ECG) signals from the skin of the user to the device. The dry electrode offers advantages over gel electrodes, including improved ease of manufacture, better storage stability, and enhanced signal stability. The ECG sensor 312 in the device can use two exposed leads on the bottom to record electrocardiogram measurements at a sampling rate of 100 Hz. This rate is lower than most medical-grade ECG systems but is sufficient for capturing all necessary data for athletic and most medical uses. The electrocardiogram sensor 312 can include an instrumentation amplifier with analog filtering to enhance signal quality. The ECG sensor 312 can be configured to reduce common mode noise interference without the need for a third electrode by approximating the impedance of the skin and adding the inverted common mode signal directly into the existing leads. The system 100 can calculate heart rate from the ECG sensor 312. The process can involve bandpass filtering 10 seconds of ECG data to identify the R wave using a peak detection algorithm. The algorithm can also be configured to reject noise introduced by motion.

The three-axis accelerometer 318 (e.g., MEMSIC® MC3479 accelerometer) of the system 100 can be configured for monitoring external factors such as user motion and impacts. Three-axis accelerometer 318 can operate at a sampling rate of 100 Hz and can be configured to monitor acceleration in several ranges from +/−2 g to +/−16 g, allowing for flexibility depending on the specific use case.

The three-axis magnetometer 320 (e.g., MEMSIC® MMC5603NJ magnetometer) can be included in the system 100 to measure magnetic fields, primarily to enhance the reliability of the acceleration measurements. This sensor operates at a sampling rate of 1 Hz and is used to correct for any drift or error in the accelerometer data, ensuring more accurate motion tracking.

The device is equipped with two temperature sensors 314, 316 can be positioned to measure both the skin temperature via the first temperature sensor 314 and the ambient temperature around the device 302 via the second temperature sensor 316.

In certain embodiments, the system 100 can be used to calculate and monitor various physiological and biomechanical metrics. Metrics such as heart rate, external workload, external energy expenditure, and temperature values can be calculated via the calculation module 306.

External workload can be calculated as an accumulation of changes in acceleration for an athlete, combining three-dimensional movement into a single value. The metric can be calculated from acceleration and magnetometer data. One second of acceleration data is filtered to remove the gravity vector, complemented by the magnetometer measurement to reduce error and sensor drift. The difference of the filtered acceleration signal is then summed to calculate the external workload over a single second. This data is streamed to the cloud through the relay collector system, and the total external workload data can be accumulated to calculate a total value for the athlete for the duration of their workout. Both instantaneous (e.g., measured at the one second interval) workload and total external workload can be displayed by the team dashboard.

The system 100 can calculate core body temperature, which can be an important metric for monitoring the health and performance of athletes during physical activities. The calculation can be facilitated by an algorithm that can integrate machine learning and/or artificial intelligence techniques to enhance accuracy and efficiency. The use of machine learning means that the algorithm is not static; it adapts and improves over time. The algorithm can learn from each prediction it makes, adjusting its model parameters to reduce errors in future predictions. This adaptive learning process can be supported by continuous data collection, allowing the model to be always evolving and improving its accuracy and efficiency. The algorithm not only collects and averages data but can also employ advanced data handling techniques to filter out noise and irrelevant information, which allows the prediction to be based on the most relevant and accurate data available.

A long-short term memory (LSTM) model, a type of recurrent neural network (RNN) known for its ability to process sequences of data, is an example of an acceptable type of algorithm that can be employed for this purpose. Unlike standard neural networks, LSTMs have feedback connections that make them capable of processing entire sequences of data. This means the algorithm can remember information for long periods, which is crucial for understanding the context in a sequence of physiological data points. This memory feature allows the algorithm to see not just where the core body temperature of the athlete is at a given moment, but how it got there, which enhances prediction accuracy.

The ability of the algorithm to predict future body temperature within the predetermined interval represents an application of real-time predictive analytics. This short-term forecasting allows for immediate interventions that can be important during sports activities, where rapid responses to physiological changes can militate against injuries and optimize performance. The predictions generated by the algorithm can be used to adjust the model parameters continuously. This adaptive learning process allows the algorithm to improve its predictions over time, learning from each cycle of data collection, processing, and prediction. This feature is particularly important in dynamic and variable conditions such as sports activities, where physiological responses can vary significantly between individuals and across different conditions.

The algorithm can begin by collecting real-time physiological data from sensors embedded in the wearable device. These sensors continuously monitor the heart rate of the athlete, skin and ambient temperature of the athlete, and the duration and intensity (e.g., determined by external workload and heart rate) of physical activity. This continuous data collection can provide a comprehensive dataset that reflects the current physiological state of the athlete. These variables can be measured continuously at a predetermined interval, with the heart rate and skin temperature data being averaged over intervals ranging from 2 to 30 seconds, for example. The algorithm can process data from the previous 2 to 20 minutes to forecast the core body temperature 0 to 60 seconds into the future, ensuring timely and accurate readings. This averaging can help to smooth out fluctuations in the data caused by transient factors, providing a more stable and reliable dataset for analysis.

External energy can be calculated to measure how much energy (e.g., calories) the user expends through external movement. External energy can be calculated with the same signal as external workload—the filtered acceleration value. The calculation can involve determining a change in kinetic energy.

A total energy expenditure of the user can be calculated by combining external and internal energy measurements. External energy is derived as described above, while internal energy can be calculated using heat transfer principles. The device can measure core body temperature, skin temperature, ambient temperature (e.g., inside the clothes of the user), and environmental temperature. These temperatures can allow for the calculation of the temperature difference, thereby enabling the determination of heat transfer and the measurement of energy expended as heat is transferred from the core of the body to the environment.

These metrics can provide insights into the performance and health of athletes, enabling coaches and medical staff to make informed decisions to optimize training and ensure safety. The ability to calculate and analyze these metrics in real-time represents a significant advancement in sports technology and athlete monitoring.

The system 100 can be utilized in a system specifically configured for use by teams of athletes. In team mode, the communication module 308 can transmit data, which is collected by the system 100. Data from all athletes utilizing one of the devices can be uploaded to the cloud-based system and accessed by coaches, trainers, medical staff, or any other approved users in real time. The system 100 can include a charging case 400 that can hold and charge multiple devices simultaneously, streaming all the data to the cloud.

The system 100 can include a user interface on the device that displays important metrics directly, enabling users to receive immediate feedback. Additionally, the system 100 can store historical data of physiological parameters, which can be accessed to analyze trends and changes over time. This historical data is important for adjusting training programs and monitoring the effectiveness of fitness regimes.

The system 100 can include an associated dashboard that serves as a component for data visualization and analysis. The dashboard can be configured to display real-time physiological and biomechanical data collected from athletes, which can be accessed by coaches, trainers, medical staff, and other authorized users. The primary functionality of the dashboard is to provide immediate feedback and adjustments during training sessions or competitions by presenting real-time data such as heart rate, external workload, and temperature measurements.

One of the features of the dashboard is its ability to monitor and issue alerts regarding potential safety concerns. For instance, it can alert users if an athlete's physiological readings suggest a risk of health issues, enabling timely interventions. This proactive monitoring helps in militating against injuries and managing health risks effectively.

Additionally, the dashboard facilitates the access and analysis of historical data, which is invaluable for assessing the progress and effectiveness of training programs over time. Coaches and trainers can use this data to make informed decisions about adjustments to training regimens, ensuring optimal performance and recovery for athletes.

The dashboard can be customizable to display various metrics tailored to the specific needs or preferences of the team or individual users, enhancing its utility and adaptability. Integration with other software systems used by the team is possible, allowing for a comprehensive management approach that can include training schedules, athlete health records, or nutritional tracking systems. This integration provides a holistic toolset for effective athlete management, making the dashboard an essential part of the monitoring system.

It has been advantageously found that the employment of the first temperature sensor for determining skin temperature, and the second temperature sensor for determining ambient air temperature, permits for the effective calculation of the core body temperature which, heretofore, was only able to be determined by invasive temperature probes. The employment of the biocompatible adhesive together with this unique use of temperature sensors, which serves to minimize loss of contact and undesirable shifting of the sensors, also facilitates this determination that was not otherwise known prior to the present disclosure.

While certain representative embodiments and details have been shown for purposes of illustrating the present disclosure, it will be apparent to those skilled in the art that various changes may be made without departing from the scope of the disclosure, which is further described in the following appended claims.

What is claimed is:

1. A system for monitoring physiological and biomechanical parameters of a user, comprising:
an affixable, wearable housing, the housing including;
a primary electronics body disposed at least partly within the housing, the primary electronics body;
a first temperature sensor operably connected to the primary electronics body to measure skin temperature of the user;
an electrocardiogram sensor operably connected to the primary electronics body;
a communication module operably connected to the primary electronics body configured to continuously receive data from the first temperature sensor and a heart rate derived from the electrocardiogram sensor the electrocardiogram sensor;
a calculation module operably connected to the communication module, the calculation module configured, using data from the first temperature sensor and the electrocardiogram sensor from the communication module, to calculate a core body temperature of the user.

2. The system of claim 1, wherein the calculation module is configured to calculate the core body temperature using an algorithm stored in the memory, the algorithm using the data from the first temperature sensor, the heart rate derived from the electrocardiogram sensor, and the duration and the intensity since the user began exercising.

3. The system of claim 1, wherein the algorithm uses data collected over a predetermined interval.

4. The system of claim 1, further comprising a second temperature sensor positioned within the housing to measure ambient temperature around the housing, wherein ambient temperature data is to monitor heat transfer as a predictor of heat related illness.

5. The system of claim 1, further comprising a three-axis accelerometer configured to monitor external factors including motion and impacts of the user.

6. The system of claim 5, wherein the calculation module is further configured to calculate external workload and external energy expenditure based on data from the accelerometer.

7. The system of claim 5, further comprising a three-axis magnetometer.

8. The system of claim 1, wherein the housing includes a biocompatible adhesive configured to removably affix the housing to the skin of the user.

9. The system of claim 1, wherein the communication module includes a transmitter for transmitting the calculated core body temperature to a mobile device or computer system for real-time monitoring.

10. The system of claim 1, wherein the core body temperature is continuously calculated every 10 seconds to provide monitoring during physical activity.

11. The system of claim 1, wherein the system is configured to store historical data of physiological parameters of the user, allowing for trend analysis over time to monitor changes in the user's health or fitness level.

12. The system of claim 1, further comprising a charging case configured to store and charge the affixable wearable housing.

13. A system for calculating external workload of a user, comprising:
an affixable, wearable housing;
a primary electronics body disposed at least partly within the housing;
a three-axis accelerometer positioned within the housing configured to sample acceleration values and monitor external factors including motion and impacts of the user;
a three-axis magnetometer positioned within the housing configured to measure magnetic fields to enhance the accuracy of the accelerometer measurements;
a communication module positioned within the housing configured to transmit data to a cloud-based system;
a calculation module operably connected to the communication module, wherein the calculation module is configured to calculate external workload, by:
filtering one second of acceleration data to remove a gravity vector using both accelerometer and magnetometer data;
calculating a difference of the filtered acceleration signal;
summing the differenced signal to calculate the external workload data over a single second.

14. The system of claim 13, wherein the calculated external workload data is streamed to the cloud-based system via the communication module for further analysis and display.

15. The system of claim 14, wherein the external workload data is accumulated to calculate a total external workload value for a duration of a workout of the user.

16. The system of claim 13, wherein the external workload calculation is used to adjust a training program of the user automatically.

17. The system of claim 13, further comprising a charging case configured to store and charge the affixable wearable housing.

18. The system of claim 17, further comprising multiple wearable housings disposed in the charging case.

19. A system for calculating energy expenditure of a user, comprising:
an affixable, wearable housing;
a primary electronics body disposed at least partly within the housing;
a three-axis accelerometer positioned within the housing configured to continuously sample acceleration values and monitor external factors including motion and impacts of the user;
a temperature sensor array including sensors for measuring skin temperature, ambient temperature around the housing, and environmental temperature positioned within the housing;
a communication module positioned within the housing configured to transmit data to a cloud-based system;
a calculation module operably connected to the communication module, wherein the calculation module is configured to calculate energy expenditure by:
calculating external energy using data from the accelerometer calculating a change in kinetic energy, which considers force, displacement, and an angle of motion;
calculating internal energy using data from the temperature sensor array by determining heat transfer based on the temperature difference between a core body temperature, a skin temperature, an ambient temperature, and environmental temperatures; and
summing the calculated external and internal energy values to determine total energy expenditure data of the user.

20. The system of claim 19, wherein the energy expenditure data is transmitted to the cloud-based system via the communication module for further analysis and display.
